(19) United States
(12) Patent Application Publication
(10) Pub. No.: US 2013/0272962 A1
(43) Pub. Date: Oct. 17, 2013

(54) STAINING AGENT FOR CORNEAL STAINING

(71) Applicant: FLUORON GMBH, Ulm (DE)

(72) Inventor: Wilfried Kugler, Ulm (DE)

(21) Appl. No.: 13/861,259

(22) Filed: Apr. 11, 2013

(30) Foreign Application Priority Data
Apr. 11, 2012 (DE) 102012103097.9
Nov. 9, 2012 (DE) 102012110745.9

(51) Int. Cl. A61K 49/00; G01N 1/30

(52) U.S. Cl. CPC A61K 49/00; G01N 1/30
USPC 424/9.1; 435/29

(57) ABSTRACT

The present invention relates to a staining agent for dyeing an ophthalmic membrane, in particular the cornea of a human eye or an animal's eye. In particular, the invention relates to the use of an aqueous, physiologically compatible solution, in which a dyestuff has been dissolved, for the purpose of coloring membranes, in particular the cornea or parts thereof in the human eye or in the eye of an animal.

### CROSS REFERENCE TO RELATED APPLICATIONS

This application claims priority under 35 U.S.C. §119(a) to German (DE) Application No. DE 10 2012 110 745.9 having a filing date of Nov. 9, 2012 and to German (DE) Application No. DE 10 2012 103 097.9 having a filing date of Apr. 11, 2012, which applications are incorporated by reference in their entirety.

### BACKGROUND

1. Technical Field

The present invention relates to a staining agent for dyeing an ophthalmic membrane, in particular the cornea of a human eye or an animal's eye. In particular, the invention relates to the use of an aqueous, physiologically compatible solution, in which a dyestuff has been dissolved, for the purpose of coloring membranes, in particular the cornea or parts thereof, in the human eye or in the eye of an animal.

2. Description of the Related Art

The use of vital dyestuffs in order to examine the viability of endothelial cells in donor corneas, for example from donor banks, has long been known (A comparison of two different staining methods for evaluating corneal endothelial viability. Stocker F. W. et al., Arch Ophthalmol. 1966; 76(6): 833-835). The advantage of vital staining over other procedures is the rapid and simple determination of endothelial cells in the cornea that have died.

For the vital staining of donor corneas, in many cornea banks trypan blue is used; this compound, known from the class of the diazo dyestuffs, colors the nucleus of damaged or dead endothelial cells in the donor corneas (Evaluation of the endothelium of human donor corneas by induced dilation of intercellular spaces and trypan blue. Sperling S., Graefe's Arch Clin. Exp. Ophthalmol. 1986; 224(5): 428-434). However, the reliability of staining with trypan blue in cases of endothelial cytometry has been called into question (Value of two mortality assessment techniques for organ cultured corneal endothelium: trypan blue versus TUNEL technique. Gain P. et al., Br. J. Ophthalmol. 2002; 86(3): 306-310).

In the case of trypan blue it is a question of a cytotoxic substance, as known, for example, from Veckeneer M. et al.: Ocular toxicity study of trypan blue injected into the vitreous cavity of rabbit eyes, Graefe's Arch Clin. Ex. Ophthalmol. (2002) 239: 698-704 and Rezai K. A. et al.: Trypan Blue Induces Apoptosis in Human Retinal Pigment Epithelial Cells, Am. J. Ophthalmol. (2004) 138: 492-495.

A further rapid and simple vital staining for the coloring of donor corneas is offered by the test with Janus green, which colors the cell nuclei and mitochondria of damaged cells (A new test for endothelial viability. The Janus green photometry technique. Hartmann C. et al.; Arch. Ophthalmol. 1989; 107(10): 1511-1515).

The vital staining with indocyanine green for the purpose of determining the viability of endothelial cells of donor corneas has likewise long been known (Indocyanine green: a new vital stain for use before penetrating keratoplasty. McEnerney J. K. et al., Arch Ophthalmol. 1978; 96(8): 1445-1447) but has been unable to win through against the use of trypan blue.

The dyestuff alizarin red has been tested in laboratory studies as an addition to trypan blue in dual stainings and has been postulated as useful for the purpose of distinguishing living and dead cell boundaries (Dual staining of corneal endothelium with trypan blue and alizarin red S: importance of pH for the dye-lake reaction. Taylor M. J. et al., Br. Ophthalmol. 1981; 65(12): 815-819).

A further dyestuff that is used in the examination of the vitality of donor corneas is fluorescein diacetate; the latter likewise colors dead corneal endothelial cells (Differential value of various vital stains of corneal endothelium. Wilhelm F. et al., Ophthalmologe. 1995; 92(4): 496-498). This dyestuff, however, has also been unable to win through against the use of trypan blue.

Rose bengal was regarded for decades as a dyestuff that is able to color epithelial cells of the cornea that have died. But more recent investigations have shown that rose bengal is also capable of penetrating into intact cells and then colors them (Patient tolerance and ocular surface staining characteristics of lissamine green versus rose bengal. Manning et al., Ophthalmology 1995; 102: 1953-1957). Rose bengal has proved in the past to be cytotoxic. The cytotoxicity is intensified additionally by interaction with light (Evaluation of the Effect of Lissamine Green and Rose Bengal on Human Corneal Epithelial Cells, Kim J. et al., Cornea 1999; 18: 328-332).

Stained structures also permit, besides the above application in connection with the determination of the vitality of donor corneas, a better visualization during a surgical intervention, and contribute to lowering distinctly the operational risk for the patient, and to enhancing the post-operative outcome distinctly. New techniques in corneal-transplantation surgery, such as, for example, anterior lamellar keratoplasty (DALK=deep anterior lamellar keratoplasty) or posterior lamellar keratoplasty (DLEK=deep lamellar endothelial keratoplasty), such as, for example, DMEK (Descemet's membrane endothelial keratoplasty) and DSAEK (Descemet's stripping with automated endothelial keratoplasty), result in better surgical outcomes. These lamellar techniques, however, presuppose a precise determination of the individual corneal layers. Vital dyestuffs can assist or enable the identification of these layers by virtue of their special coloring, inasmuch as they are able to stain specific structures such as, for example, the collagen fibers of the stroma, the endothelial cells or Descemet's membrane in the donor cornea and also in the recipient cornea.

In keratoplasty, trypan blue has been employed hitherto in the case of penetrating and deep lamellar keratoplasty (Use of trypan blue for penetrating keratoplasty. Roos J. C.; J. Cataract Refract. Surg. 2005; 31(10); 1867-1869; Deep lamellar keratoplasty with trypan blue intrastromal staining. Balestrazzi E.; J. Cataract Refract. Surg. 2002; 28(6): 929-931). Trypan blue has been postulated as a sensible intraoperative staining agent, in order to make posterior stromal layers visible.

As already described herein, trypan blue is a cytotoxic substance. In connection with the use of trypan blue, therefore, complete irrigation, particularly of the ocular region in which the trypan blue has come into operation as staining agent, is necessary immediately after the operation, in order to avoid a lengthy stay in the body or, to be more precise, in the eye.

A further dyestuff that has been tested in the DLEK procedure is indocyanine green (ICG). In this case the corneal stroma of the donor, which is to be transplanted, has been stained (Use of indocyanine green in deep lamellar endothelial keratoplasty. John T.; J. Cataract Refract. Surg. 2003; 29(3):437-443).

However, it is known that ICG is phototoxic (Phototoxicity of Indocyanine Green on Human Retinal Pigment Epithelium in Vitro and its Reduction by Lutein. Wu W-C. et al., Photochemistry and Photobiology 2005; 81: 537-540) and cytotoxic (Comparative studies on the retinal toxicity of trypan blue and indocyanine green. Ito T. et al., Invest. Ophthalmol Vis. Sci. 2004; 45: 3669-B130).

Gentian violet has been proposed as a staining agent for the peripheral stromal surface with Descemet's membrane and for the endothelium in the DLEK procedure (Simple technique to unfold the donor corneal lenticule during Descemet's stripping and automated endothelial keratoplasty. Koenig S. B., J Cataract Refract. Surg. 2007; 33(2): 189-190). Gentian violet, however, is known to cause corneal oedemas (Gentian violet solution for staining the anterior capsule. Ünlü K. et al.; J. Cataract Refract. Surg. 2000; 26: 1228-1232, and Experimental staining of the anterior lens capsule in albino rabbits, Gamal Eldin et al.; J. Cataract Refract. Surg. 1999; 25: 1289-1294).

For the purpose of coloring an incision into a clear cornea, the use of a blade coated with trypan blue has been proposed, in order to make the incision more capable of being visualized. (Clear corneal incision with trypan-blue-coated blades. Kayikcioglu O.; J. Cataract Refract. Surg. 2007; 33(2): 351-352).

However, dyestuffs that are used conventionally in order to carry out the vital staining of donor corneas or that have been postulated in order to be employed in surgical procedures on the cornea, such as, for example, procedures of transplantation of the cornea, continue to exhibit disadvantages in cytotoxicity, staining behavior, production and/or use. Reports concerning potential toxicity, teratogenicity etc., technical problems such as solubility, and problems in use—such as complicated staining techniques, pigmenting behavior and irrigating behavior—still make the use thereof in corneal surgery a difficult field.

The previous dyestuffs that have been used sometimes have, as already stated, an unfavorable safety profile, so that up until today none of these dyestuffs has been authorized for use in corneal surgery. A further consideration sometimes is a certain non-specificity of the dyestuffs. In order not to be dependent on a single dyestuff, it is sensible to search in parallel for further alternatives, possibly dyestuffs that are more compatible.

By reason of this, there is a desire for the development of a staining agent for ophthalmic surgery, in particular for corneal staining, or of a staining agent for improving ophthalmic surgery, particularly in connection with corneal staining, whereby this staining agent is desirably to exhibit good staining properties paired with high biocompatibility/lack of cytotoxicity.

### DESCRIPTION OF THE INVENTION

The object of the invention is therefore the provision of a staining agent that complies with the ideas and needs in the state of the art that were described above. This staining agent is to be suitable for making visible ophthalmic membranes, in particular the cornea or parts thereof or constituents thereof.

This object is achieved, in accordance with the invention, by the invention described in the present application, in particular by the features of the independent claims. The dependent claims and the present application further provide preferred embodiments of the invention.

As defined in the claims, a biocompatible staining agent for staining the cornea or parts thereof or constituents thereof is made available that contains as principal component at least one dyestuff that has been selected from triphenylmethane dyestuffs and/or azo dyestuffs, cyanine dyestuffs, naphthocyanine dyestuffs, as described in WO 2011/151287, and/or natural dyestuffs, or a pharmaceutically compatible salt and/or hydrate of these dyestuffs, as well as a pharmaceutically acceptable carrier. The staining agent according to the invention is produced by dissolving the dyestuff in an aqueous solution. The aqueous solution preferentially contains a buffer, for example a hydrogenphosphate buffer consisting of disodium hydrogenphosphate and sodium dihydrogenphosphate, sodium chloride and water.

Surprisingly, it has been found that the use of Acid Violet 17 in a staining agent for staining ophthalmic membranes presents not only an alternative but a multilayered improvement in comparison with the state of the art. In particular, it has been found that the staining agent according to the invention stains both ophthalmic membranes to be removed from the eye and the anterior and posterior lens capsules, and in addition is also capable of staining the cornea or constituents thereof. This joint staining capacity could not be expected, since hitherto it had not been observed in any known staining agent. The same holds for Brilliant Blue G (BBG). Therefore the two dyestuffs Acid Violet 17 and BBG, particularly BBG, but also generally biocompatible triphenylmethane dyestuffs, are preferred dyestuffs in the sense of the invention.

In addition, it has surprisingly been found that Acid Violet 17 is very readily soluble in liquids that are suitable for application both in the anterior region and in the posterior region of the eye, such as, for example, balanced salt solution (BSS), water for injection, etc. Besides, Acid Violet 17 is also very readily soluble in liquids that are heavier than water, that is to say, ≧1.01 g/cm³, such as, for example, polyethylene glycol. The good solubility of Acid Violet 17 means that a dyestuff solution according to the invention can be readily produced, because no small lumps or flocculations—that is to say, insoluble dyestuff crystals/particles—arise. The same holds for BBG.

The use of the staining agent according to the invention in a procedure of optical surgery described herein is also capable of being managed correspondingly well. In such a procedure it is extraordinarily important that in the course of application, particularly in the course of applying the dyestuff, no small lumps are formed and no flocculations occur, since otherwise the cannula of the syringe would become clogged with the staining agent, as a result of which the operating surgeon would exert higher pressure, so that in the worst case the staining agent would have to be administered with excessive pressure which ultimately has the result that cells, tissue and/or structures in the eye would be damaged or even destroyed. By virtue of insoluble dyestuff crystals/particles that get into the patient's eye during the operation and possibly remain there, furthermore mechanical and/or immunological problems might also arise.

The preferred good solubility of the staining agent according to the invention also has the result that the staining agent can be washed out readily. A good capacity to be washed out is important, since excess staining agent should be removed from the eye in order to rule out or prevent any possible late trauma or other complications as far as possible.

The staining qualities of BBG and Acid Violet 17 are improved in comparison with the dyestuffs used at present, such as trypan blue (ERM staining, capsular staining) or Blueron (capsular staining), indocyanine green (ILM staining); consequently, as described herein, both BBG and Acid Violet 17, respectively, i.e., a triphenyl methane dye can stain objects at very low dyestuff concentrations. The improvement in comparison with the state of the art is due to an improved visualization, a faster coloring of the ophthalmic membranes, and also a higher-contrast staining at low concentration.

In addition, it has been observed that Acid Violet 17 is preferentially capable of coloring both the inner limiting membrane (ILM) and the anterior and posterior lens capsules. The same holds for BBG. Hitherto no dyestuff has been known that in the case of both ophthalmological membranes displays a corresponding coloring, capable of being used in ophthalmic surgery, and consequently permits a possibly simultaneous coloring of the membranes.

Furthermore, the staining agent according to the invention can additionally be used for coloring the cornea or the layers of the cornea described herein, for example in the case of transplantations of the cornea. This means that in one embodiment the staining agent according to the invention is able to color the inner limiting membrane (membrana limitans interna, ILM), the anterior and/or posterior lens capsule and/or the cornea or constituents thereof.

The present invention therefore provides a biocompatible staining agent for use in a procedure for staining the cornea, or for use in a procedure of corneal surgery, for example keratoplasty.

The present invention further also provides the use of the biocompatible staining agent for the purpose of staining the cornea, as well as the use of the biocompatible staining agent for the purpose of producing a medical product or diagnostic agent for staining the cornea or for use in a procedure of corneal surgery, for example keratoplasty.

A further aspect of the present invention is a biocompatible staining agent for use in an ophthalmological or optical-surgery procedure (procedure of optical surgery or surgical procedure of ophthalmology), particularly in corneal surgery. The procedure of optical surgery preferentially includes the coloring of the cornea and the implementing of a procedure of optical surgery described herein, in particular corneal surgery, for example a procedure of keratoplasty, for example corneal transplantation surgery such as, for example, anterior lamellar keratoplasty (DALK=deep anterior lamellar keratoplasty) or posterior lamellar keratoplasty (DLEK=deep lamellar endothelial keratoplasty) such as, for example, DMEK (Descemet's membrane endothelial keratoplasty) and DSAEK (Descemet's stripping with automated endothelial keratoplasty).

Another aspect of the present invention is a procedure for coloring the cornea, comprising coloring the cornea and implementing a surgical procedure, described herein, of ophthalmology. A preferred surgical procedure is transplantation of the cornea, comprising removing and/or inserting at least one part of a cornea or parts thereof or constituents thereof or the entire cornea, stained with the staining agent according to the invention. In this connection the cornea or parts/regions of the cornea of the donor and/or the cornea or parts/regions of the cornea of the recipient can be colored with the staining agent according to the invention, and in this way the surgical procedure on and/or with the colored cornea can be implemented.

A yet further aspect of the present invention is the coloring of the cornea with the staining agent according to the invention, said cornea being located in a cornea bank, in order in this way to discover the orientation of the cornea and/or to verify the orientation of the cornea.

An important constituent of the preparation according to the invention is the dyestuff. By way of dyestuff, compounds can be employed that are able to color the cornea or constituents thereof specifically and selectively, so that the cornea or constituents thereof differ visually from other structures. Moreover, the dyestuff must be soluble in water or in the mixture consisting of water and a further solvent. Said dyestuff must neither be toxic, in particular cytotoxic or cytopathic, nor cause damage to the cornea, for example oedemas (as does gentian violet, also called gentian blue), and/or to the retina, or produce toxic effects by virtue of light reactions, as does ICG or trypan blue. In addition, said dyestuff should have a good staining power, in order to be able to keep the quantity of the dyestuff small.

Dyestuffs from the group of the triphenylmethane dyestuffs, such as Acid Violet 17, Brilliant Blue G (BBG), Brilliant Blue R (BBR), Brilliant Blue FCF, Patent Blue V, bromophenol blue, Fast Green, Methyl Green, Acid Brilliant Green, rosanilin; from the group of the azo dyes and diazo dyes, such as Orange G, Ponceau 2R, Chromotrope 6R, Ponceau 6R, tartrazine, azophloxine, Ponceau B 1, Evans blue, Chicago blue; from the group of the cyanine dyestuffs, such as 3,3′-diethylthiacyanine iodide, 3,3′-diethylthiacarbocyanine iodide, 3,3′-diethyl-9-methylthiacarbocyanine iodide, 1,1′-diethyl-4,4′-cyanine iodide; and/or from the group of the natural dyestuffs, such as orcein, lawsone, indigotin, canthaxanthin, haematoxylin, indigo carmine, anthocyans, anthocyanidins, lutein, zeaxanthin and/or anthraquinones as well as mixtures thereof, or mixtures of several members of one of the stated groups as well as members of different groups, have proved advantageous.

Use is preferably made of triphenylmethane dyestuffs. Of the triphenylmethane dyestuffs, BBG or Acid Violet 17 is particularly preferred.

Acid Violet 17 is a triphenylmethane dyestuff that serves as a dyestuff in the textile industry. In addition, it is utilized in biochemistry for the purpose of coloring proteins in the course of polyacrylamide gel electrophoresis.

“Acid Violet 17” is also designated as “42650” or “Coomassie Violet”, with “Coomassie Violet” comprising Coomassie Violet R200 and R150. Acid Violet 17 has the empirical formula C41H45N3O6S2 and a preferred molecular weight of 739.94 g/mol. The CAS number is 4129-84-4. The further designations of Acid Violet 17 may be used as an alternative to the term Acid Violet 17 in connection with the present invention. Acid Violet 17 can be obtained from Sigma Aldrich, for example.

Acid Violet 17 has the following structural formula:

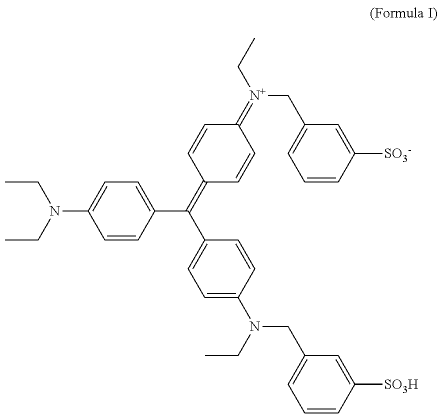

The sodium salt of Formula I is preferred:

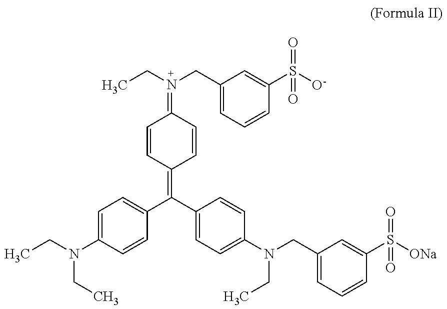

Each of these two structural formulae (Formulae I and II) may also be used as an alternative to the term “Acid Violet 17” in connection with the present invention.

The Acid Violet 17 which is used in accordance with the invention, or its pharmaceutically compatible salt and/or hydrate thereof, can be produced by processes known in the literature.

Triphenylmethane dyestuffs according to the invention are derivatives of the colorless triphenylmethane. The following grouping, as a common structural feature and chromophore of triphenylmethane dyestuffs, can be derived from triphenylmethane:

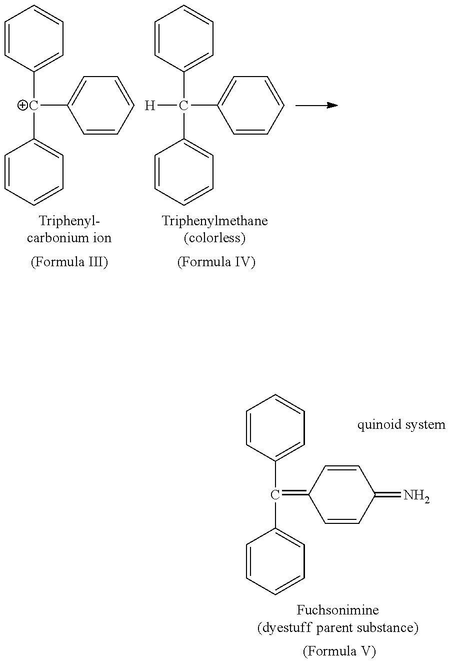

At least two of the three phenyl substituents have been substituted in triphenylmethane dyestuffs by electron-supplying auxochromic or antiauxochromic groups. As a result, the pi-electron system extends over all three benzene nuclei and the central sp2-hybridised carbon atom, forming a quinoid system. As a result, an extended mesomeric pi-electron system then arises which extends over all three rings and forms the actual parent substance of the dyestuff, the fuchsonimine. Depending on the auxochromes and antiauxochromes which are present as substituents on the individual nuclei, individual nuclei are more strongly or more weakly involved in the mesomerism, whereby as a result the pi-electron system is variably influenced and the color of the various compounds is changed. Each of the three phenyl substituents of the triphenylmethane core structure may optionally contain one or more further substituents.

In the case of auxochromic groups it is a question of substituents with free electron pairs on the chromophoric system, which with a +M effect participate in the mesomerism of the electron system and consequently bring about a further extension of the pi-electron cloud. By virtue of the increased delocalization, the electrons can be excited still more readily. Under the influence of the substituents, a shift of the absorption towards the longer wavelength takes place, and a deepening of the color (bathochromic shift) is obtained. In the order of decreasing color intensification, this is, inter alia: —NR2, —NHR, —NH2, —OR, -halogen, and —OH. Besides the deepening of the color, the action of auxochromic groups also brings about a bonding of the dyestuff to the staining material.

A further delocalization of the pi-electrons can be caused additionally by substituents on the mesomeric system which act as electron acceptors—such as, for example, C═O, —NO2 and N═N—and withdraw electrons from the pi-electron system. Such a substituent is called antiauxochromic, since it acts against an auxochromic group.

In accordance with a preferred embodiment of the present invention, for the purpose of coloring the cornea or constituents thereof in the course of corneal surgery a biocompatible staining agent containing a dyestuff selected from triphenylmethane dyestuffs and/or azo dyestuffs and/or cyanine dyestuffs and/or natural dyestuffs such as anthocyans and anthocyanidins, and/or a pharmaceutically compatible salt and/or hydrate thereof, is provided as principal component.

Examples of pharmaceutically compatible salts include salts with inorganic bases, ammonia, organic bases, inorganic acids, organic acids, basic amino acids, halogen ions or such like, as well as inner molecular salts. Examples of an inorganic base include alkali metal (e.g., Na, K) and alkaline-earth metal (e.g., Ca, Mg). Examples of the organic base include trimethylamine, triethylamine, choline, procaine, ethanolamine and such like. Examples of the inorganic acid include hydrochloric acid, hydrobromic acid, sulfuric acid, nitric acid and phosphoric acid and such like. Examples of the organic acid include p-toluenesulfonic acid, methanesulfonic acid, formic acid, trifluoroacetic acid, maleic acid and such like. Examples of the basic amino acid include lysine, arginine, ornithine, histidine and such like. The compound may also be a pharmaceutically acceptable hydrate.

In accordance with a preferred embodiment of the present invention, the staining agent can be utilized as a surgical auxiliary agent in the course of ophthalmological operations relating to ophthalmic diseases such as, for example, diseases of the vitreous body and of the retina, such as macular hole, retinal detachment due to a highly myopic eye, epiretinal membrane, proliferative diabetic retinopathy, macular oedema such as, for example, diabetic macular oedema, and proliferative vitreoretinopathy, as well as specific cataracts such as hypermature cataract and congenital cataract; furthermore, it can also be utilized in the case of penetrating keratoplasty, anterior and posterior lamellar keratoplasty and corneal marking, etc. By reason of the colored composition of the present invention, it becomes possible to make more clearly visible the ophthalmic membranes that can only be recognized with difficulty, and to enhance safety during surgical interventions.

In another preferred embodiment of the present invention the staining agent can be utilized in order to stain an ophthalmic membrane, particularly preferably in order to stain the inner limiting membrane, the anterior and posterior lens capsules and/or the cornea or constituents thereof.

The ophthalmic membrane may also be the cornea of the eye, but it is not necessarily limited to this. In the case of the staining of the cornea the situation is such that the performing operating surgeon is able to mark the cornea of the eye of the transplant recipient, in order then to excise appropriate sites, for example mechanically or by means of excimer-laser trepanation, and to replace them with a graft. In the case of the transplantation of the cornea, all the layers of the cornea may be transplanted (penetrating keratoplasty), or only special layers of the cornea (lamellar keratoplasty). In order now to make visible the cornea or regions or sites both of the donor cornea and of the acceptor cornea, use may be made of the staining agent according to the invention, in order, for example, to mark the cornea from outside or, in the case of the suturing of the donor cornea to the recipient cornea, to serve as suture marker.

In accordance with a more preferred embodiment of the present invention, the staining agent can be utilized as a surgical auxiliary agent in the course of ophthalmological operations such as, for example, thermokeratoplasty, penetrating keratoplasty, anterior and posterior lamellar keratoplasty and corneal marking, epikeratophakia etc., relating to ophthalmic diseases such as, for example, diseases of the cornea such as corneal dystrophies (e.g., Reis-Bücklers corneal dystrophy and Fuchs corneal endothelial dystrophy), bullous keratopathies (e.g., pseudophakic bullous keratopathy and keratopathy in the case of pseudoexfoliation syndrome), corneal inflammations or keratitis (e.g., metaherpetic keratitis), superficial corneal scars after injuries, infections or caustic burns of the cornea, impending perforation in the case of brittle or thinned corneal tissue, tumors growing exophytically and pterygia, tissue defects after removal of dermoids of the cornea in children. By reason of the colored composition of the present invention, it becomes possible to make more clearly visible the structures of the cornea that can only be recognized with difficulty, and to enhance safety during surgical interventions.

An “ophthalmic membrane” is a membrane in the eye or, to be more exact, such a membrane that delimits structures in the eye and/or separates them from one another and therefore constitutes a cellular boundary layer in the eye. There are a large number of such boundary layers. They include, for example, Bruch's membrane, which is a limiting membrane between the choroid and the retinal pigment epithelium (RPE) of the eye, the basal membrane, in which connection all the epithelial tissues rest on a basal membrane which separates them from the underlying connective tissue; Descemet's membrane, which is located between the corneal stroma and the corneal endothelium and is also known by the designation ‘lamina limitans posterior’; Bowman's membrane, also Bowman's layer or lamina limitans anterior, which lies between the corneal stroma and the basal membrane of the corneal endothelium and is about 12 μm thick; the vitreous limiting membrane, an extremely fine membrane surrounding the gel-like vitreous body and bordering the lens at the front and otherwise the retina, and being detached from these in old age; epiretinal membrane, also known as macular pucker or as cellophane retinopathy, are pathological (diseased) membrane formations which are characterized by the development of a membrane above the macula (central retina); the capsular sack of the lens, which constitutes the basal membrane of the epithelial cells of the lens and encloses the crystalline lens; the conjunctiva, which is the transparent membrane that seals off our open eye; Barkan's membrane, which is a persistent mesodermal tissue in the iridocorneal angle in the case of congenital glaucoma; the pseudomembrane, which consists of cells as a consequence of chronic conjunctivitis; the table-top membrane, which is seated on the macula; the inner limiting membrane (ILM, membrana limitans interna), this being, as inner boundary layer of the retina, the basal membrane of the Müller cells of the retina; the outer limiting membrane (ELM, membrana limitans externa), which is the outer boundary layer of the retina and is located between the pigment epithelium and the photoreceptors (rods and cones) of the retina; the secondary membrane, which is a secondary cataract or even postoperative cataract, in which connection it is a question of a fibrotic change of the posterior lens-capsule membrane which is left behind in the eye in the case of cataract surgery; the inflammatory membranes, which are protein layers that have arisen by virtue of inflammatory processes and that may be stratified upon numerous structures in the eye, for example lens, synthetic lens, posterior lens capsule after cataract operation, ciliary body, etc.

Ophthalmic membranes that are preferred in accordance with the invention are the retina, the anterior and posterior lens capsules, the inner limiting membrane (membrana limitans interna, ILM), the epiretinal membrane (ERM) or the cornea. The cornea is built up from various layers, in particular from an epithelium (=uppermost covering layer), a first intermediate membrane (Bowman's membrane or lamina limitans anterior), the main substance (=stroma or corneal parenchyma), a second intermediate membrane (=Descemet's membrane or lamina limitans posterior), and the lowest covering layer (=endothelium). These layers are also encompassed by the term “ophthalmic membrane”.

“Membranes to be removed from the eye” or “a membrane to be removed from the eye” means an ophthalmic membrane, preferentially the inner limiting membrane (membrana limitans interna, ILM), the epiretinal membrane (ERM), the anterior and posterior lens capsules and/or the cornea or constituents thereof.

With a view to removing the epiretinal membrane, the adjacent retinal tissue or ILM is stained by the staining agent according to the invention. When the membrane is removed from the underlying non-dyed retinal tissue, a good contrast then results. After the coloring, excess staining-agent solution is rinsed out, and the free space is filled up by BSS or, in ablation surgery, by air, gas or silicone oil. By virtue of the dyeing, it is possible to work with an unilluminated or only weakly illuminated instrument in the course of abrading (peeling) the membrane. By this means, with sufficient perception of contrast the light toxicity is considerably reduced. Particularly in the case of application in connection with epiretinal membranes (epiretinal gliosis, macular pucker, surface wrinkling), the use of the staining agent according to the invention constitutes a valuable aid when seeking out and removing the membranes.

The terms “dyeing,” “coloring” or “staining,” when they are used herein, can be used synonymously. Each of the terms implies that an ophthalmic membrane is either itself stained/colored by the staining agent according to the invention (so-called positive staining) and/or that the ophthalmic membrane itself is in fact not stained, but rather ophthalmic membranes that are situated around the ophthalmic membrane that itself is not stained and/or that adjoin the ophthalmic membrane that itself is not stained (so-called negative staining). A negative staining arises, for example, in the case of the ERM if use is made of the staining agent according to the invention. The terms also imply that structures outside the eye as described herein are also colored.

The cornea is built up from various layers, in particular from an epithelium (=uppermost covering layer), a first intermediate membrane (Bowman's membrane or lamina limitans anterior), the main substance (=stroma or corneal parenchyma), a second intermediate membrane (=Descemet's membrane or lamina limitans posterior), and the lowest covering layer (=endothelium). These layers are defined herein as parts or constituents of the cornea. By the term “cornea”, the entire cornea and/or parts thereof or constituents thereof are meant.

In another preferred embodiment of the present invention the staining agent can be utilized in order to stain one or more constituents of the cornea; particularly preferred in this case is the coloring of the stroma, for example the peripheral stromal surface, Descemet's membrane and the endothelium.

A staining agent, a pharmaceutically compatible salt and/or hydrate thereof as principal component can likewise be utilized outside the eye for the purpose of staining sclera, conjunctiva, tendons, muscles and fibrotic tissue, for example in connection with the treatment of squinting (strabismus) or of posterior tenectomy, and also for the purpose of staining Tenon's capsule, for example in the course of enucleation.

A staining agent, a pharmaceutically compatible salt and/or hydrate thereof can furthermore be used for the purpose of coloring medicaments such as, for example, antiproliferative agents in connection with operations on the eye, such as, for example, in connection with trabeculectomy in the region of the anterior chamber.

Moreover, the present invention provides the use of the dyestuffs described herein for the purpose of producing a staining agent for the visualization or coloring of ophthalmic membranes.

In addition, according to a further main aspect of the present invention the use of the dyestuffs as surgical auxiliary agent for ophthalmological operations is made available.

In a preferred embodiment of the present invention it is preferred that the staining agent contains the dyestuff in a concentration of 1×10⁻³-10 g/L; particularly preferred is a concentration from 0.1 g/L to 1.5 g/L for the staining of the cornea or constituents thereof. In this embodiment a staining agent with a high staining affinity at low concentrations and in small quantities is provided, and a spontaneous dyeing of the desired regions in the human eye or in the eye of an animal is obtained.

In accordance with one embodiment of the present invention, it is preferred that the staining agent is present in a physiologically compatible aqueous solution of, in particular, sodium chloride, which can be adjusted to a pH from 6.8 to 7.8, in particular about 7.4, with a buffer. By way of buffer, use may be made of a phosphate buffer, carbonate buffer or citrate buffer, the pH value of which can be adjusted by means of sodium hydroxide. The solution may preferably be an intraocular irrigating solution, a balanced salt solution or a physiological common-salt solution.

The preparation according to the invention is based, in one embodiment, on water by way of solvent, whereby further solvents may, where appropriate, be contained in small proportions, provided they are homogenously miscible with water and are biologically compatible. Here, monohydric and polyhydric alcohols such as also find application in the medical field enter into consideration. If use is made of a further solvent, this is particularly preferably a glycol or glycerine. Mixtures of the stated solvents also enter into consideration. If a solvent is admixed to the water, said solvent should be used in a proportion of not more than 20 wt %, more preferably not more than 10 wt %.

Besides water by way of solvent and the dyestuff, the staining agent according to the invention contains, where appropriate, an agent adjusting the density. The agent adjusting the density must be biocompatible, must not be toxic and must be homogeneously miscible with water, where appropriate after addition of a small quantity of a solubility promoter such as alcohol, so that a clear transparent solution arises. In addition, said agent must be compatible with the dyestuff, i.e., it must not impair the solubility of the dyestuff to a considerable extent.

In another preferred embodiment of the present invention the density of the staining agent is therefore adjusted within a range from 1.01 g/cm³ to 1.50 g/cm³, preferably 1.01 g/cm³ to 1.30 g/cm³. The range between 1.001 g/cm³ and 1.01 g/cm³ is also envisaged, for example 1.001 g/cm³, 1.002 g/cm³, 1.003 g/cm³, 1.004 g/cm³, 1.005 g/cm³, 1.006 g/cm³, 1.007 g/cm³, 1.008 g/cm³, 1.009 g/cm³. The osmolarity should furthermore lie within a range of 280-330 mosmol/L and should preferably amount to 300 mosmol/L. Liquids compatible with water, the density of which lies above the density of water, enter into consideration. The density is preferentially measured at 20° C., i.e., the temperature of the staining agent should amount to 20° C., the density being measured at this temperature. The density is preferentially measured with a pycnometer. A preferred pycnometer may be, for example, a Mettler Toledo DA-100M Density Meter (RBE93409) (range: 0-3 g/cm³, precision: 0.001 g/cm³).

Agents for adjusting the density are liquids compatible with water, the density of which lies above the density of water. An advantageous agent for increasing the density is heavy water, D₂O, with which the density value can be adjusted to the desired range. Heavy water is distinguished by outstanding compatibility; it is tolerated by eukaryotes up to a concentration of 20% in water and does not result in irritations in the field of application; it is miscible with water in any concentration, does not have a tendency to settle or separate, and with respect to solubility exhibits no detectable differences in comparison with water. The proportion of heavy water in the preparation can be adjusted in such a way that the desired density value from 1.01 g/cm³ to 1.50 g/cm³, preferably 1.01 g/cm³ to 1.30 g/cm³, is obtained. The suitable quantity, which also depends on the further ingredients, can be found by simple tests or calculations. If heavy water is the agent adjusting the density, it is preferably used in a quantity of 5-20% V/V, particularly preferably in a quantity of 13% V/V. The production of the preparation with heavy water is also very simple and, by reason of the good miscibility of the two constituents, can easily be effected by mixing. From water, heavy water and dyestuff it is therefore possible to produce, simply and quickly, a preparation that is stable in the long term and well-suited for the purpose of selective coloring of the membranes.

A further agent with which the density can be adjusted is a disaccharide or polysaccharide. Polysaccharides are suitable for increasing the density and are readily available. In addition, they are toxicologically unobjectionable and biocompatible. By the term “polysaccharides”, molecules are understood here that have been synthesized from more than two, preferably more than five, particularly preferably more than ten, saccharide units. Although generally monosaccharides and disaccharides can increase the density, in accordance with the invention non-reducing disaccharides are preferably employed for the purpose of increasing the density. The use of monosaccharides and reducing disaccharides may result in undesirable effects; for instance, they may be cytotoxic in the quantity necessary for increasing the density. Nonetheless, glucose is also provided as agent for adjusting the density in the sense of the invention, but then preferably in a concentration of up to (and including) 5% (v/v), more preferably in a concentration of 4.9, 4.8, 4.7, 4.6, 4.5, 4.4, 4.3, 4.2, 4.1 or 4.0% (v/v). Non-reducing disaccharides that are suitable in accordance with the invention are sucrose or trehalose. By way of suitable polysaccharides, soluble starch derivatives such as hydroxyethyl starch and dextran may be named. Mannitol is also provided. By way of polysaccharides, compounds are suitable that are neutral, act in non-reducing manner and do not decompose in aqueous solution.

Further agents for adjusting the density are neutral polymers such as polyethers, for example polyethylene glycol (PEG), polyvinyl alcohol, polyester, polyacrylic acid copolymer, polyvinylpyrrolidone. Similarly, hyaluronic acid or derivatives of hyaluronic acid, such as esters thereof, is/are provided for the purpose of adjusting the density. Combinations of the stated agents are also well-suited in order to adjust the density of the preparation according to the invention, for example a combination of heavy water and one or more polysaccharides. The quantity of heavy water and/or further or other agents adjusting the density is chosen in such a way that the density of the finished preparation lies within the requisite range from 1.01 g/cm³ to 1.50 g/cm³, preferably 1.01 g/cm³ to 1.30 g/cm³. The density of the preparation can be determined by any common method such as is generally known to a person skilled in the art.

In another preferred embodiment of the present invention the density of the staining agent is adjusted within a range from 0.50 g/cm³ to 0.99 g/cm³, preferably 0.80 g/cm³ to 0.99 g/cm³. The osmolarity should furthermore lie within a range of 280-330 mosmol/L and should preferably amount to 300 mosmol/L. Liquids compatible with water, the density of which lies below the density of water, enter into consideration. An advantageous agent for lowering the density is, for example, alcohol in physiologically compatible quantities, with which the density value can be adjusted to the desired range. The range between 1.001 g/cm³, and 1.01 g/cm³ is also provided as density for the staining agent, for example 1.001 g/cm³, 1.002 g/cm³, 1.003 g/cm³, 1.004 g/cm³, 1.005 g/cm³, 1.006 g/cm³, 1.007 g/cm³, 1.008 g/cm³, 1.009 g/cm³.

It is further provided that in a preferred embodiment of the invention the staining agent is mixed with a viscoelastic substance, or a viscoelastic substance is added to the staining agent. The viscosity of such a staining agent preferentially amounts to at least 1.5 mPa·s or 2.0 mPa·s, more preferably 2.2 mPa·s, 2.3 mPa·s, 2.4 mPa·s or 2.5 mPa·s. The viscosity preferentially amounts to less than 18 mPa·s; more preferably it amounts to less than 9 mPa·s, 8 mPa·s, 7 mPa·s, 6 mPa·s or 5 mPa·s. The viscosity is preferentially measured at a temperature of 20° C., i.e., the staining agent should have a temperature of 20° C. in the course of measurement.

Ophthalmological operations on and/or with and/or with involvement of the cornea are, for example, the penetrating and lamellar procedures of keratoplasty described herein. In this connection the cornea or parts thereof is/are colored with the staining agent according to the invention, and then the appropriate operation is carried out thereon as described herein.

For example, in connection with the staining of the cornea the situation is such that the performing operating surgeon is able to mark the cornea, or parts thereof, of the eye of the transplant recipient (transplant acceptor) and/or of the transplant donor, in order then to excise appropriate sites, for example mechanically or by means of excimer-laser trepanation, and to replace them with a graft. In the course of the transplantation of the cornea, all the layers of the cornea may be transplanted (penetrating keratoplasty), or only special layers of the cornea (lamellar keratoplasty). In order now to make the cornea or regions or sites both of the donor cornea and of the recipient cornea visible, use may be made of the staining agent according to the invention, in order, for example, to mark the cornea from outside or, in the case of the suturing of the donor cornea to the recipient cornea, by way of suture marker.

Ophthalmological or surgical procedures (procedures of optical surgery) as described herein include, in particular, keratoplasty, comprising thermokeratoplasty, penetrating keratoplasty and lamellar keratoplasty. The latter is employed in modern ophthalmology in connection with a large number of specific, disabling corneal diseases. Most corneal grafts are carried out for optical reasons, i.e., in order to eliminate opacities of the normally optically clear cornea. A keratoplasty may furthermore be necessary in order to eliminate active seats of infection of the corneal tissue or to offset defects such as occur, for example, after severe inflammations, injuries or operations. In most cases the integrity of the remaining structures of the eye is preserved, and a good visual rehabilitation is possible.

In the case of the lamellar techniques, in principle a distinction is made between anterior and posterior lamellar keratoplasties, which can be employed in indication-specific manner.

In the case of anterior lamellar keratoplasty (DALK=deep anterior lamellar keratoplasty) the trepanation of the recipient cornea is not performed completely but only so far as the deep layers of the stroma. Subsequently the dissection is carried out, wherever possible, as far as Descemet's membrane, so that the eye is not opened up and the layer of cells that is crucial for the prognosis, the endothelium of the recipient, remains. The insertion of the donor corneal disc is effected after removal of the endothelium and of Descemet's membrane. This procedure is consequently only suitable if the endothelium of the recipient is intact. A classical indication is constituted by keratoconus. The deep dissection of the corneal lamella in the recipient can also be performed in pachymetry-controlled manner with an excimer laser (PALK=pachymetry-assisted laser keratoplasty).

Posterior lamellar keratoplasty is indicated if the epithelium and stroma of the patient are intact but an endothelial disease is present. Classical indications are Fuchs endothelial dystrophy and bullous keratopathy as a consequence of a decompensation of the endothelium after a cataract operation. There are two basic techniques: DSAEK (Descemet's stripping with automated endothelial keratoplasty) and DMEK (Descemet's membrane endothelial keratoplasty).

In the case of the DSAEK technique, in an artificial anterior chamber a thin posterior stromal lamella with intact endothelium is obtained from the donor cornea with the aid of a microkeratome. This specimen is brought into the anterior chamber of the recipient and is placed there onto the cornea from inside after the endothelium there has previously been removed.

The DMEK method is employed in order to transplant exclusively the endothelium with Descemet's membrane. In this connection, only Descemet's membrane with the endothelium is dissected away from the donor cornea, in order then to be introduced and applied in the anterior chamber of the recipient, in which the corneal endothelium has previously been removed. This requires a great deal of patience and skill on the part of the operating surgeon.

Overall, fundamental advantages of the lamellar procedures over penetrating keratoplasty might be expected: lower intraoperative risk, faster visual rehabilitation, less irregular development of astigmatism, fewer rejection reactions, a lower transplant-failure rate, longer survival-time of the transplants, and a lower re-keratoplasty rate. However, the lamellar techniques are associated with a longer learning-curve and with greater effort in terms of time and equipment; in addition, they are less standardized than penetrating keratoplasty.

According to a further aspect of the present invention, the use of the staining agent as a surgical auxiliary agent for ophthalmological operations on or with involvement of the cornea is made available.

Moreover, the present invention describes the use of a dyestuff described herein, in particular a triphenylmethane dyestuff for producing a biocompatible staining agent according to the invention for coloring the cornea and/or as auxiliary agent for coloring the cornea in the course of ophthalmological operations as described herein on and/or with the cornea.

In a preferred embodiment a staining agent according to the invention includes two or more dyestuffs selected from the group consisting of triphenylmethane dyestuffs, azo dyestuffs, natural dyestuffs, cyanine dyestuffs and/or naphthocyanine dyestuffs, as described in WO 2011/151287, for example.

The staining agent according to the invention is preferentially a medical product which preferentially includes a pharmaceutically compatible carrier. The medical product is preferentially present in aqueous form. As an alternative, said medical product may also be present in freeze-dried form and may be reconstituted as needed.

The term “pharmaceutically compatible” or “pharmaceutically acceptable” used herein implies that the corresponding substance is physiologically compatible and also biocompatible and remains in the staining agent according to the invention in the course of the ophthalmological interventions described herein without injurious influence on the patient.

In comparison with the traditional trypan blue, which has a teratogenic or mutagenic effect (Cahen R L: Evaluation of the teratogenicity of drugs, Clin Pharmacol. Ther, 1964, 5, 480-514 and product information BLURHEX™, Dr. Agarwal's Pharma Ltd. Chemai (India)), ICG, which is phototoxic (Phototoxicity of Indocyanine Green on Human Retinal Pigment Epithelium in Vitro and its Reduction by Lutein, Wu W.-C. et al., Photochemistry and Photobiology 2005; 81: 537-540) and cytotoxic (Comparative studies on the retinal toxicity of trypan blue and indocyanine green, Ito T. et al., Invest. Ophthalmol. Vis. Sci. 2004; 45; 3669-B130), and also gentian violet, which causes corneal oedemas (Gentian violet solution for coloring the anterior capsule, Ünlü K. et al.; J Cataract Refract Surg. 2000; 26; 1228-1232 and Experimental coloring of the anterior lens capsule in albino rabbits, Gamal Eldin et al.; J. Cataract Refract. Surg. 1999; 25; 1289-1294), the biocompatible solution according to the invention possesses no cytotoxicity.

For the purpose of demonstrating the lack of cytotoxicity, mouse cells, preferentially L929 cells (ATCC No. CCL1, NCTC clone 929), were brought into contact with the staining agent according to the invention with varying concentrations of dyestuff (e.g., starting from a 0.6 mg/ml solution, dilutions are prepared such as, e.g., 13.2%, 19.8%, 29.6%, 44.4%, 66.7%, and the original solution with 100%) over about 68 hours to 72 hours in an incubator at about 37° C. and with 5% CO₂ in accordance with the prescribed cytotoxicity test (Standard series ISO 10993). The cells are preferentially cultured in culture vessels, in particular cell-culture flasks with an area of, preferentially, 75 cm² or 175 cm², and in these culture vessels are also brought into contact with the staining agent and cultivated. The culture medium preferentially contains a culture medium known to a person skilled in the art, such as, for example, DMEM (e.g., from Invitrogen), also 10% FCS (foetal calf serum), for example 10% FCS Gold from PAA. The inhibition of growth is determined by processes known as such. The vitality of the cells and a derived cytotoxicity are determined quantitatively by determination of the protein content of the treated cell cultures in comparison with untreated control cultures. With a standard procedure, preferentially BCA staining, the protein content is ascertained colorimetrically. In this connection it becomes evident that cytotoxicity at a significant level corresponding to an inhibition of growth of more than 30% in comparison with L929 cells that were not brought into contact or cultivated with the staining agent is not present. By way of positive control, use may be made, for example, of medium (e.g., DMEM) with FCS, for example 10% and 5% DMSO with the corresponding test cells, in order to see whether the test cells are inhibited in their growth. By way of negative control, use is made of test cells with medium, for example DMEM with 10% FCS, that is to say, no dyestuff solution added.

Additionally or alternatively, the lack of cytotoxicity of the staining agent can be evaluated on the basis of ARPE 19 cells with varying concentrations of dyestuff (e.g., 0.5 g/L, 0.25 g/L and 0.1 g/L). The exposure-times on the cell cultures preferentially amount to 30 seconds, 60 seconds, 120 seconds and 300 seconds. Dual determinations are preferentially carried out. The evaluation is preferentially effected by means of an MTT assay. For this purpose the rows of cells are preferentially brought to 100% confluence in 12-well plates. Per well, 1 mL of dissolved dyestuff is applied in each case in the corresponding time. After the application of dyestuff, the cells are given a period of 24 hours for an appropriate reaction. Then the MTT assay is carried out and evaluated in an ELISA reader. Per cell line and per solvent, preferentially three mutually independent series of tests are carried out.

The staining agent according to the invention preferentially causes no corneal oedema as does, for example, gentian violet (gentian blue), see Ünlü et al. or Gamal Eldin et al., both cited above.

The term “lack of toxicity”, “biocompatibility” or “biocompatible” implies that the staining agent according to the invention in the prescribed cytotoxicity test (e.g., according to standard series ISO 19993) using the specified quantities/concentrations in the case of L929 cells causes no inhibition of growth of more than 30% under the described conditions in comparison with untreated L-929 cells, and using the specified quantities/concentrations in the case of ARPE-19 cells causes no inhibition of growth of more than 20% under the described conditions in comparison with untreated ARPE-19 cells. By virtue of the high biocompatibility of the staining agent there are justified grounds for the assumption that side effects known hitherto, such as oedemas for example, can be prevented.

The staining agent according to the invention is accordingly preferentially used in a concentration that in the tests described herein displays no cytotoxicity and yet can still bring about a sufficient staining of the cornea. A person skilled in the art is capable of determining the staining on the basis of known test processes.

A biocompatible staining agent as defined in the Claims is made available that contains as principal component at least one dyestuff that has been selected from triphenylmethane dyestuffs and/or azo dyestuffs and/or cyanine dyestuffs and/or natural dyestuffs, or a pharmaceutically compatible salt and/or hydrate of these dyestuffs, for the purpose of staining the cornea or constituents thereof, as well as a pharmaceutically acceptable carrier.

The terms “dyeing”, “coloring” or “staining”, when they are used herein, can be used synonymously. Each of the terms implies that an ophthalmic membrane, in particular the cornea, either is itself stained/colored by the staining agent according to the invention (so-called positive staining) and/or that the ophthalmic membrane, in particular the cornea, is itself not stained but rather ophthalmic membranes that are situated around the ophthalmic membrane that itself is not stained, in particular the cornea, and/or that adjoin the ophthalmic membrane that itself is not stained, in particular the cornea (so-called negative staining). The coloring may relate to the entire ophthalmic membrane or to parts of the ophthalmic membrane, in particular of the cornea, for example the marking of certain regions of the cornea or so-called corneal tattooing.

In accordance with one embodiment of the present invention, the staining agent for staining the cornea or constituents thereof is made available at the time of removal and/or prior to the time of removal of a part of the cornea or of a part of constituents thereof.

The staining agent can be used for coloring the corneal stroma, in particular for the peripheral stromal layer of the donor cornea in the case of a DLEK operation. As a result, the contact surface between donor cornea and recipient cornea is made visible for the operating surgeon, positively assisting the operation. Furthermore, it is ensured that the layers of the donor cornea are incorporated into the recipient cornea in the correct orientation, in order consequently to guarantee the success of the operation.

The staining agent can be used for coloring Descemet's membrane of the cornea, particularly in the case of a penetrating keratoplasty. By virtue of the staining of the donor membrane and/or recipient membrane, the adaptation of the cut edges of the donor membrane and recipient membrane can be assisted, resulting in an improvement of the stability of the graft and also in a diminution of an astigmatism caused by the operation. Additionally in the case of penetrating keratoplasty, residues of Descemet's membrane can be made visible by the staining agent after removal of the recipient cornea, and can be removed from the eye in order to minimize complications after the intervention.

The staining agent can be used for coloring the endothelium of the cornea, particularly in the course of the transplantation of Descemet's membrane. As a result, it is once again ensured that the layers of the donor cornea are incorporated into the recipient cornea in the correct orientation, in order consequently to guarantee the success of the operation.

The use of the staining agent according to the invention can positively influence the use of halogen light and xenon light during the surgical intervention. In particular, the dyestuff used in the staining agent is, in contrast to ICG, not phototoxic.

Moreover, the invention provides a procedure for the ex vivo staining of the human cornea or the cornea of an animal, or parts thereof or constituents thereof, comprising staining the removed cornea or parts thereof or constituents thereof with a staining agent containing at least one dyestuff that has been selected from triphenylmethane dyestuffs, azo dyestuffs, cyanine dyestuffs, naphthocyanine dyestuffs and/or natural dyestuffs. The staining agent preferentially stains the stroma of the cornea or parts thereof.

Furthermore, it is possible to dye a viscoelastic material, for example hyaluronic acid which comes into operation as an auxiliary agent in connection with ophthalmological surgery, with the aqueous staining-agent solution. Preferentially a triphenylmethane dyestuff, for example Acid Violet 17 or BBG, or an azo dyestuff is mixed with a viscoelastic material such as, for example, hyaluronic acid or derivatives thereof such as ester for example. Preferentially by means of the hyaluronic acid the density as herein is adjusted in such a way that the solution is heavier than water, for example heavier than 1.001 g/cm³, preferentially not heavier than 1.01 g/cm³, for example 1.002 g/cm³, 1.003 g/cm³, 1.004 g/cm³, 1.005 g/cm³, 1.006 g/cm³, 1.007 g/cm³, 1.008 g/cm³, 1.009 g/cm³.

Furthermore, with the staining agent according to the invention it is also possible to stain a different physiologically compatible liquid that is used in ophthalmic surgery. These liquids may be or may contain, in particular, semifluorinated alkanes and/or perfluorinated carbons.

Moreover, the staining agent according to the invention may also be used for the purpose of coloring ophthalmic liquids such as, for example, vitreous-body tamponades, silicone oils or semifluorinated alkanes and/or perfluorinated carbons (see EP 859 751 or WO 2011/151079). Accordingly, the present invention provides an ophthalmic liquid that has been added to the staining agent according to the invention, and a process for producing a colored ophthalmic liquid, comprising adding the ophthalmic liquid to the staining agent according to the invention and obtaining the colored ophthalmic liquid. The ophthalmic liquid may be, for example, a viscoelastic liquid to be used in connection with an eye operation.

With the staining agents according to the invention it is possible to carry out, besides the simple stainings already described, also multiple stainings or negative stainings, in order, for example, to ensure the completeness of the removal of a membrane from the eye.

According to the colored composition of the present invention, it is made possible to make ophthalmic membranes that are difficult to recognize more clearly visible, and to enhance safety during the operation.

The staining agent may be presented in the form of a kit with a solvent and active-substance powder, or as a solution which has preferentially been filled into a syringe. Mostly it is preferred that it is provided in the form of a solution, but it is not restricted thereto. The kit is preferentially used in a procedure of optical surgery as described herein. The staining agent of the kit is preferentially presented filled in a container or in a syringe.

With respect to a further aspect of the present invention, a composition as defined in the claims is made available for use in a procedure for staining and removing and/or inserting the cornea or constituents thereof, said procedure having the steps of producing a staining agent that contains at least one dyestuff that has been selected from triphenylmethane dyestuffs and/or azo dyestuffs and/or cyanine dyestuffs and/or natural dyestuffs, or a pharmaceutically compatible salt and/or hydrate of these dyestuffs, as principal component; staining the cornea or constituents thereof, utilizing a previously determined concentration of this staining agent; and removing and/or inserting at least one part of the stained cornea or constituents thereof.

For one embodiment of the present invention, for the purpose of staining the cornea or constituents thereof any method can be used that is readily comprehensible to a person skilled in the state of the art, for example application, injection, infusion and/or irrigation.

With respect to a further main aspect of the present invention, a use of the staining agent according to the invention for the purpose of treating ophthalmic diseases, particularly of the cornea, as described herein is made available.

In addition, according to a further main aspect of the present invention the use of the staining agent according to the invention as a surgical auxiliary agent for ophthalmological operations, in particular ophthalmological operations on the cornea as described herein, is made available.

Moreover, the staining agent and/or the use thereof in a staining procedure of the present invention is utilized as part of an ophthalmological operation. Accordingly, the present invention also relates to a procedure for staining the cornea or parts thereof or constituents thereof, comprising preparing a staining agent according to the invention and staining the cornea or constituents thereof in the case of transplantation of the cornea, for example penetrating keratoplasty, anterior and posterior lamellar keratoplasty, corneal marking etc.

The staining agent according to the invention is preferentially a medical product which preferentially includes a pharmaceutically compatible carrier. The medical product is preferentially present in liquid form. As an alternative, said medical product may also be present in freeze-dried form and may be reconstituted as needed.

The procedure also includes, where appropriate, the preparing of a staining agent according to the invention before the staining as described herein is carried out.

Moreover, the present invention also relates to a procedure for staining and removing membranes to be removed from the eye, in particular the ophthalmic membranes in the case of retinal or vitreous-body surgery, comprising staining one or more membranes to be removed from the eye, in particular the ophthalmic membranes in the case of retinal or vitreous-body surgery, and removing the membranes to be removed, in particular the ophthalmic membranes in the case of retinal or vitreous-body surgery. Removal is preferentially effected by so-called peeling.

In accordance with a preferred embodiment of the present invention, in the case of the ophthalmological operations it is a question of operations for treating macula hole, retinal detachment due to a highly myopic eye, epiretinal membrane, proliferative diabetic retinopathy, macular oedema such as, for example diabetic macular oedema, and proliferative vitreoretinopathy, as well as specific cataracts such as hypermature cataract and congenital cataract; furthermore, the inventive staining agent can also be utilized in the case of penetrating keratoplasty, anterior and posterior lamellar keratoplasty and corneal marking etc.

Moreover, the ophthalmological operations described herein according to a preferred embodiment of the present invention are carried out on eyes of mammals such as, for example, dogs, cats, horses and, more preferably, on eyes of human beings.

Further aspects of the invention are:

1. Staining agent for coloring an ophthalmic membrane, containing Acid Violet 17, a pharmaceutically compatible salt and/or hydrate thereof and a pharmaceutically acceptable carrier.

2. Staining agent according to aspect 1, wherein the ophthalmic membrane is the inner limiting membrane and/or the anterior and/or posterior lens capsule and/or the cornea or constituents thereof.

3. Staining agent according to aspect 1 or 2, wherein the staining agent contains Acid Violet 17 in a concentration of 1×10⁻³-10 g/L, preferably in a concentration of 0.005-1.5 g/L and most preferably in a concentration of 0.10-0.50 g/L, for the staining of the ILM, in a concentration from 0.20 g/L to 1.0 g/L for the staining of the lens capsule, and also in a concentration from 0.1 g/L to 1.5 g/L for the staining of the cornea or constituents thereof.

4. Staining agent according to one of aspects 1 to 3, wherein the pharmaceutically acceptable carrier is a physiologically compatible aqueous solution with a pH from 6.8 to 7.8.

5. Staining agent according to one of the preceding aspects, wherein the density of the staining agent is adjusted within a range from 1.01 g/cm³ to 1.50 g/cm³, preferably 1.01 g/cm³ to 1.30 g/cm³.

6. Staining agent according to aspect 5, wherein the agent adjusting the density is D₂O.

7. Staining agent according to one of the preceding aspects, wherein the density of the staining agent is adjusted within a range from 0.50 g/cm³ to 0.99 g/cm³, preferably 0.80 g/cm³ to 0.99 g/cm³.

8. Staining agent according to one of the preceding aspects, wherein the staining agent exhibits an osmolarity of 280-330 mosmol/L, preferably 300 mosmol/L.

9. Staining agent for use in a procedure of optical surgery comprising the dyeing of the ophthalmic membrane and the implementation of the removal of the ophthalmic membrane, wherein the staining agent comprises Acid Violet 17, a pharmaceutically compatible salt and/or hydrate thereof and a pharmaceutically acceptable carrier.

10. Staining agent according to aspect 9, wherein the ophthalmic membrane is the inner limiting membrane and/or the anterior and/or posterior lens capsule and/or the cornea or constituents thereof.

11. Staining agent according to aspect 9 or 10, wherein the dyestuff contains Acid Violet 17 in a concentration of 1×10⁻³-10 g/L, preferably in a concentration of 0.005-1.5 g/L and most preferably in a concentration of 0.10-0.50 g/L, for the staining of the ILM, in a concentration from 0.20 g/L to 1.0 g/L for the staining of the lens capsule, and also in a concentration from 0.10 g/L to 1.5 g/L for the staining of the cornea or constituents thereof.

12. Staining agent according to one of aspects 9 to 11, wherein the pharmaceutically acceptable carrier is a physiologically compatible aqueous solution with a pH from 6.8 to 7.8.

13. Staining agent according to one of aspects 9 to 12, wherein the density of the staining agent is adjusted within a range from 1.01 g/cm³ to 1.5 g/cm³, preferably 1.01 g/cm³ to 1.3 g/cm³.

14. Staining agent according to aspect 13, wherein the agent adjusting the density is D₂O.

15. Staining agent according to one of aspects 9 to 12, wherein the density of the staining agent is adjusted within a range from 0.50 g/cm³ to 0.99 g/cm³, preferably 0.80 g/cm³ to 0.99 g/cm³.

16. Staining agent according to one of aspects 9 to 15, wherein the staining agent exhibits an osmolarity of 280-330 mosmol/L, preferably 300 mosmol/L.

17. Kit containing a solvent and Acid Violet 17, a pharmaceutically compatible salt and/or hydrate thereof as active-substance powder, or containing a staining agent as defined in aspects 1 to 8, which is preferentially presented filled into a syringe.

18. Kit according to aspect 17 for use in a procedure of optical surgery according to one of aspects 9 to 16.

19. Medical product, comprising the staining agent defined as in aspects 1 to 16.

The various embodiments described above can be combined to provide further embodiments. All U.S. patents, U.S. patent application publications, U.S. patent applications, foreign patents, foreign patent applications and non-patent publications referred to in this specification and listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

I claim the following:

1. A biocompatible staining agent for coloring the cornea or parts thereof or constituents thereof, comprising at least one dyestuff that has been selected from triphenylmethane dyestuffs and/or azo dyestuffs and/or cyanine dyestuffs and/or naphthocyanine dyestuffs and/or natural dyestuffs, or a pharmaceutically compatible salt and/or hydrate of these dyestuffs, as well as a pharmaceutically acceptable carrier.

2. The biocompatible staining agent according to claim 1, wherein the dyestuff is a triphenylmethane dyestuff or a pharmaceutically compatible salt and/or hydrate thereof.

3. The biocompatible staining agent according to claim 2, wherein the dyestuff is BBG or Acid Violet 17 or a pharmaceutically compatible salt and/or hydrate thereof.

4. The biocompatible staining agent according to claim 1, wherein the dyestuff is present in a concentration of 1×10⁻³-10 g/L, preferably in a concentration from 0.1 g/L to 1.5 g/L.

5. The biocompatible staining agent according to claim 1, wherein the pharmaceutically acceptable carrier is a physiologically compatible aqueous solution with a pH from 6.8 to 7.8.

6. The biocompatible staining agent according to claim 1, wherein the staining agent exhibits an osmolarity of 280-330 mosmol/L, preferably 300 mosmol/L.

7. A method of optical surgery, in particular corneal surgery, comprising the dyeing of the cornea or parts thereof or constituents thereof and the implementation of the removal and/or insertion of at least one part of the stained cornea or constituents thereof or of the entire cornea, using a biocompatible staining agent comprising at least one dyestuff that has been selected from triphenylmethane dyestuffs and/or azo dyestuffs and/or cyanine dyestuffs and/or naphthocyanine dyestuffs and/or natural dyestuffs, or a pharmaceutically compatible salt and/or hydrate of these dyestuffs, and, where appropriate, a pharmaceutically acceptable carrier.

8. The method according to claim 7, wherein the dyestuff is a triphenylmethane dyestuff or a pharmaceutically compatible salt and/or hydrate thereof.

9. The method according to claim 8, wherein the dyestuff is BBG or Acid Violet 17 or a pharmaceutically compatible salt and/or hydrate thereof.

10. The method according to claim 7, wherein the dyestuff is present in a concentration of 1×10⁻³-10 g/L, preferably in a concentration from 0.1 g/L to 1.5 g/L.

11. The method according to claim 7, wherein the pharmaceutically acceptable carrier is a physiologically compatible aqueous solution with a pH from 6.8 to 7.8.

12. The method according to claim 7, wherein the staining agent exhibits an osmolarity of 280-330 mosmol/L, preferably 300 mosmol/L.

13. Kit containing a solvent and a dyestuff for producing the staining agent of claim 1 as active-substance powder, or containing the staining agent of claim 1, which is preferentially presented filled into a syringe.

14. The kit according to claim 13, wherein the dyestuff is present filled in a container or in a syringe.

15. The kit according to claim 13 for use in the method of optical surgery according to claim 7.

16. A medical product, comprising the staining agent of claim 1.

17. A method for the ex vivo staining of the human cornea or the cornea of an animal, or parts thereof or constituents thereof, comprising staining the removed cornea or parts thereof or constituents thereof with a staining agent containing at least one dyestuff that has been selected from triphenylmethane dyestuffs and/or azo dyestuffs and/or cyanine dyestuffs and/or naphthocyanine dyestuffs and/or natural dyestuffs.

18. The method according to claim 17, wherein the staining agent stains the stroma of the cornea or parts thereof.
